(19) United States
(12) Patent Application Publication
(10) Pub. No.: US 2017/0182292 A1
(43) Pub. Date: Jun. 29, 2017

(54) TREATING SOLID TUMOURS WITH NK-92 CELLS APPLIED BY MICROCATHETER

(71) Applicant: NANTKWEST, INC., Culver City, CA (US)

(72) Inventor: Hans G. Klingemann, Culver City, CA (US)

(73) Assignee: NANTKWEST, INC., Culver City, CA (US)

(21) Appl. No.: 15/313,312

(22) PCT Filed: May 21, 2015
(86) PCT No.: PCT/US2015/032075

§ 371 Date: Nov. 22, 2016

Related U.S. Application Data
(60) Provisional application No. 62/002,126, filed on May 22, 2014.

(51) Int. Cl. A61M 25/00; A61K 35/17

(52) U.S. Cl. CPC A61M 25/0084; A61K 35/17

(57) ABSTRACT

Disclosed herein are methods for treating solid mass tumors with direct delivery of an anti-tumor immunotherapeutic agent to the tumor site. In one aspect, this invention encompasses methods of treating solid mass tumors by direct microinjection via a microcatheter of an anti-tumor immunotherapeutic agent into the microvasculature leading into tumor thereby providing high levels of contact with the tumor while minimizing the degree of systemic buildup of the immunotherapeutic agent.

### FIELD OF THE INVENTION

This invention relates to methods for treating solid mass tumors with direct delivery of an anti-tumor immunotherapeutic agent to the tumor site. In one aspect, this invention encompasses methods of treating solid mass tumors by direct microinjection via a microcatheter of an anti-tumor immunotherapeutic agent into the microvasculature leading into tumor thereby providing high levels of contact with the tumor while minimizing the degree of systemic buildup of the immunotherapeutic agent. In various embodiments, the solid mass tumor is a brain tumor and the microinjection of the anti-tumor agent bypasses the blood brain barrier. In other embodiments, the anti-tumor immunotherapeutic agent comprises one or more cell lines used in immunotherapy of tumors such as NK-92 cells.

### BACKGROUND OF THE INVENTION

Certain solid mass tumors are deemed inoperable due to their location in the body. For example, brain tumors are difficult to treat as many of the commonly used anti-tumor chemotherapeutic agents lack the ability to cross the blood brain barrier. Radiation therapy may likewise be unavailable due to the location of the tumor in the brain. While other solid mass tumors may be deemed treatable by chemotherapeutic agents, the use of such agents poses a serious risk of systemic circulation at a concentration that causes significant adverse side effects. In addition, many patients become recalcitrant to the chemotherapeutic agents leaving them without recourse to treat the cancer.

Advances in immunotherapy poses some benefits and involves the use of certain cells of the immune system that have cytotoxic activity against particular target cells. For example, natural killer (NK) cells are cytotoxic lymphocytes that constitute a major component of the innate immune system. Natural killer (NK) cells, generally representing about 10-15% of circulating lymphocytes, bind and kill targeted cells, including virus-infected cells and many malignant cells, non-specifically with regard to antigen and without prior immune sensitization. Herberman et al., Science 214:24 (1981). Killing of targeted cells occurs by inducing cell lysis. NK cells have been shown to be somewhat effective in both ex vivo therapy and in vivo treatment. NK cells used for this purpose are isolated from the peripheral blood lymphocyte (“PBL”) fraction of blood from the subject, expanded in cell culture in order to obtain sufficient numbers of cells, and then re-infused into the subject. However, such therapy is complicated by the fact that not all NK cells are cytolytic and the therapy is specific to the treated patient.

NK-92 cells have previously been evaluated as a therapeutic agent in the treatment of certain cancers. Unlike NK cells, NK-92 is a cytolytic cancer cell line which was discovered in the blood of a subject suffering from a non-Hodgkins lymphoma. NK-92 cells lack the major inhibitory receptors that are displayed by normal NK cells, but retain the majority of the activating receptors. Characterization of the NK-92 cell line is disclosed by Gong et al., 1994; and Yan et al., 1998. NK-92 cells do not, however, attack normal cells nor do they elicit an unacceptable immune rejection response. However, NK-92 cells do not cross the blood brain barrier and the concentration required to treat a solid mass tumor is challenging.

Accordingly, there still exists an ongoing need for new methods of treating or preventing solid mass tumors in patients.

### SUMMARY OF THE INVENTION

This invention is predicated on the use of microinjection of anti-tumor immunotherapeutic agents directly into a solid mass tumor such as a brain tumor. In some embodiments, the microinjection delivery involves insertion of the microcatheter through the femoral artery and positioning of the microcatheter into the microvasculature of the solid mass tumor. Ejection of the anti-tumor immunotherapeutic agent into the solid mass tumor provides for direct microinjection delivery into the tumor, which permits the use of significantly reduced amounts of the anti-tumor immunotherapeutic agent and, correspondingly, a reduced systemic impact on the patient.

In one aspect of this embodiment, there is provided a method to treat a solid tumor by the direct microinfusion of an anti-tumor immunotherapeutic agent into said tumor, which method comprises:

a) placing a microcatheter into one or more of the microvasculature arteries feeding said tumor;

b) infusing through said microcatheter said anti-tumor immunotherapeutic agent so that said immunotherapeutic agent is directed by the microvasculature into said tumor; and

c) maintaining said infusion until a sufficient amount of said anti-tumor agent has been infused so as to treat said tumor.

In some embodiments, the method comprises the direct microinfusion of an anti-tumor immunotherapeutic agent into the tumor.

A variety of solid tumors, such as tumors of the lung, pancreas, thyroid, ovary, stomach, bladder, breast, liver, kidney, or brain, can be treated by the method of this invention. In one aspect, the solid tumor is a non-pulmonary solid mass tumor. In another aspect of the invention, the solid mass tumor is a brain tumor where the anti-tumor immunotherapeutic agent is otherwise unable to reach, or to penetrate the blood brain barrier.

In another aspect of the invention, the anti-tumor immunotherapeutic agent is cytotoxic T-cells or NK-92 cells.

In some aspects of the invention, the solid mass tumor expresses one or more cancer-associated surface antigens. In some embodiments, the T cells or NK-92 cells express chimeric antigen receptors (CARs) on the surface of the cells that recognize at least one cancer-associated surface antigen expressed by the tumor cells. In some embodiments, the tumor cells are particular responsive to immunotherapy. In aspects of the invention, the solid mass tumor expresses HER-2 receptors.

### DETAILED DESCRIPTION OF THE INVENTION

Before the present compositions and methods are disclosed and described, it is to be understood that the aspects described below are not limited to specific compositions, methods, or uses as such may, of course, vary. It is also to be understood that the terminology used herein is for the purpose of describing particular aspects only and is not intended to be limiting.

### Definitions

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise.

As used herein, the term “about” means that a value may vary +/−15%, +/−10% or +/−5% and remain within the scope of the invention. For example, “an IL-2 concentration of about 200 IU/mL” encompasses an IL-2 concentration between 170 IU/mL and 230 IU/mL.

As used to describe the present invention, “immunotherapy” refers to any antibody or naturally occurring or modified NK cell or T-cell, or cell line derived from either, whether alone or in combination and which are capable of inducing cytotoxicity when contacting a cancer cell.

As used to describe the present invention, “natural killer (NK) cells” are cells of the immune system that kill target cells in the absence of a specific antigenic stimulus, and without restriction according to MHC class. Target cells may be tumor cells or cells harboring viruses. NK cells are characterized by the presence of CD56 and the absence of CD3 surface markers.

The term “endogenous NK cells” is used to refer to NK cells derived from a donor (or the patient), as distinguished from the NK-92 cell line. Endogenous NK cells are generally heterogeneous populations of cells within which NK cells have been enriched. Endogenous NK cells may be intended for autologous or allogeneic treatment of a patient.

“NK-92 cells” refer to the immortal NK cell line, NK-92, which was originally obtained from a patient having non-Hodgkin's lymphoma. For purposes of this invention and unless indicated otherwise, the term “NK-92” is intended to refer to the original NK-92 cell lines as well as NK-92 cell lines that have been modified (e.g., by introduction of exogenous genes). NK-92 cells and exemplary and non-limiting modifications thereof are described in U.S. Pat. Nos. 7,618,817; 8,034,332; and 8,313,943, all of which are incorporated herein by reference in their entireties.

As used herein, “non-irradiated NK-92 cells” are NK-92 cells that have not been irradiated. Irradiation renders the cells incapable of growth and proliferation. It is envisioned that the NK-92 cells will be irradiated at the treatment facility or some other point prior to treatment of a patient, since the time between irradiation and infusion should be no longer than four hours in order to preserve optimal activity. Alternatively, NK-92 cells may be inactivated by another mechanism.

As used to describe the present invention, “inactivation” of the NK-92 cells renders them incapable of growth. Inactivation may also relate to the death of the NK-92 cells. It is envisioned that the NK-92 cells may be inactivated after they have effectively purged an ex vivo sample of cells related to a pathology in a therapeutic application, or after they have resided within the body of a mammal a sufficient period of time to effectively kill many or all target cells residing within the body. Inactivation may be induced, by way of non-limiting example, by administering an inactivating agent to which the NK-92 cells are sensitive.

Chimeric receptors generally comprise an exogenous antibody to specific antigen on the target cell surface and an activation/stimulation domain. The term “chimeric antigen receptor” (CAR), as used herein, refers to an extracellular antigen-binding domain that is fused to an intracellular signaling domain of a cell, such as a T cell or a NK-92 cell.

As used to describe the present invention, the terms “cytotoxic” and “cytolytic”, when used to describe the activity of effector cells such as NK cells, are intended to be synonymous. In general, cytotoxic activity relates to killing of target cells by any of a variety of biological, biochemical, or biophysical mechanisms. Cytolysis refers more specifically to activity in which the effector lyses the plasma membrane of the target cell, thereby destroying its physical integrity. This results in the killing of the target cell. Without wishing to be bound by theory, it is believed that the cytotoxic effect of NK cells is due to cytolysis.

As used to describe the present invention, the term “chemotherapeutic agents” refer to conventional and well known chemical and biological (non-cellular) agents used to treat cancer and is sometimes referred to as “conventional therapy” or “conventional treatment”. Such conventional therapy includes, but is not limited to, chemotherapy using anti-tumor chemicals, radiation therapy, hormonal therapy, and the like as well as combinations thereof.

The term “cancer stem cell” as used herein refers to cells (found within tumors or hematological cancers) that possess characteristics associated with normal stem cells, specifically the ability to give rise to all cell types found in a particular cancer sample. cancer stem cells are tumorigenic (tumor-forming), and may generate tumors through the stem cell processes of self-renewal and differentiation into multiple cell types.

As used herein “endogenous” refers to any material from or produced inside an organism, cell, tissue or system.

As used herein, the term “exogenous” refers to any material introduced from or produced outside an organism, cell, tissue or system.

The term “kill” with respect to a cell/cell population is directed to include any type of manipulation that will lead to the death of that cell/cell population.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

To “treat” a disease as the term is used herein, means to reduce the frequency or severity of at least one sign or symptom of a disease or disorder experienced by a patient.

The term “therapeutic” as used herein means a treatment and/or prophylaxis. A therapeutic effect is obtained by suppression, remission, or eradication of a disease state.

The term “therapeutically effective amount” refers to an amount of an anti-tumor immunotherapeutic agent that, when administered, is sufficient to treat a solid mass tumor. The therapeutically effective amount of the anti-tumor immunotherapeutic agent will vary depending on the tumor being treated and its severity as well as the age, weight, etc., of the patient to be treated.

Titles or subtitles may be used in the specification for the convenience of a reader, which are not intended to influence the scope of the present invention. Additionally, some terms used in this specification are more specifically defined below.

### Microcatheters

The methods of this invention are practiced using microcatheters which can be commercially available or known in the art. For example, microcatheters having a distal opening of about 330 to about 500 microns are available from ev3 Inc., Irvine, Calif., USA (http://www.ev3.net/assets/003/5260.pdf, which is incorporated herein by reference in its entirety). These microcatheters are designed to deliver liquid compositions to parts of the body distal to the femoral artery including the brain. Included in these microcatheters are those with reinforced walls which are designed to withstand significant pressure during delivery of the liquid composition to the brain. These microcatheters can be employed with a system syringe interface adapter as shown by Vascular Therapies Product Catalogue, Neurovascular, 2014 Edition, at http://www.ev3.net/pdfs/product_catalog_intl.pdf, which is incorporated herein by reference in its entirety.

Together these devices allow delivery of anti-tumor immunotherapeutic agent or a composition thereof into the microvasculature of the patient including the brain. Such devices are positioned using interventional radiology protocols well established in the art. Once positioned, the methods of this invention can be practiced as described herein.

### Methods

A biologically compatible solution comprising an anti-tumor immunotherapeutic agent is delivered by the methods of this invention directly into the microvasculature of a solid mass tumor so as to maximize its concentration in the targeted tumor. Since the tumor is directly infused with the anti-tumor immunotherapeutic agent, the amount of the composition delivered is necessarily less than that which would be delivered systemically. Moreover, in the case of brain tumors, the ability of the anti-tumor immunotherapeutic agent to cross the blood brain barrier are significantly improved.

In one embodiment, a self-sealing puncture device can be used to puncture the blood vessel so as to deliver the anti-tumor agent directly into the tumor without causing ischemia. Examples of such device are described in, e.g., WO/2013/096463, entitled “Self-Sealing Catheters” which is incorporated by reference in its entirety. For example, the catheter may have an expandable ring proximate to the catheter tip. The expandable ring is retained in compressed form when inside the lumen of the catheter. Once the catheter tip punctures the vascular wall the collar expands to seal the puncture wound. Proximal to the collar is a detachment point that allows the remainder of the catheter to separate from the tip and the collar such that the tip and collar remain in place. In one embodiment, the tip, collar and remnant of the remaining catheter tip are made of biodegradable material have a predetermined lifetime in vivo. In one embodiment, the catheter is used to administer the anti-tumor immunotherapeutic agent to a bioduct of the patient's liver.

A distal lumen opening of from about 330 to about 500 microns is ample to deliver the composition comprising the anti-tumor immunotherapeutic agent. Delivery of the anti-tumor immunotherapeutic agent is accomplished by conventional means using the microcatheters.

In one aspect, a microcatheter is used in conjunction with a balloon. The balloon is placed distal to the ejection point of the catheter so as to define a limited space between the ejection point and the balloon when placed in the vasculature or a lumen (“vasculature”). The balloon can be inflated or deflated as necessary such that when inflated, efflux from the vasculature is limited. That is to say that the inflated balloon restricts blood flow out of the tumor. The anti-tumor immunotherapeutic agent is then delivered into the tumor by the microcatheter. A chemotherapeutic agent may be co-delivered. In this way, the anti-tumor immunotherapeutic agent (and optionally chemotherapeutic agent) is retained in the tumor for a period of time, and the agent is prevented (at least partially) from entering the systemic blood. After a period of time (e.g., up to about 3 minutes), the balloon is deflated. The process can be repeated as necessary to enhance the amount of immunotherapeutic agent and optionally chemotherapeutic agent that is delivered. In one embodiment, the tumor is in the liver and rather than a vasculature, a bile duct can be used as the lumen in which the microcatheter is placed.

### Immunotherapy

Cancer immunotherapy is the use of the immune system to treat cancer. Immunotherapies include antibody therapies and cell therapies. Antibody therapies are currently the most successful form of immunotherapy, with many approved treatments for a wide range of cancers. Antibodies are proteins produced by the immune system that bind to a target antigen on the surface of a cell. In normal physiology they are used by the immune system to fight pathogens. Each antibody is specific to one or a few proteins and those that bind to cancer antigens are used in the treatment of cancer. Cell surface receptors are common targets for antibody therapies and include the epidermal growth factor receptor and HER2. Once bound to a cancer antigen, antibodies can induce antibody-dependent cell-mediated cytotoxicity, activate the complement system, prevent a receptor interacting with its ligand or deliver a payload of chemotherapy or radiation; all of which can lead to cell death. There are a number of antibodies currently approved for the treatment of cancer, including, without limitation, Alemtuzumab, Bevacizumab, Brentuximab vedotin, Cetuximab, Gemtuzumab ozogamicin, Ibritumomab tiuxetan, Ipilimumab, Ofatumumab, Panitumumab, Rituximab, Tositumomab and Trastuzumab.

Cell-based immunotherapy also holds great promise for cancer treatment. For instance, cytokine-induced killer cells (i.e., CIKs) are cytotoxic immune effector cells that have become a strong candidate for a new generation of anti-tumor immune cell therapy because CIKs have anti-tumor cytotoxicity and diverse T cell receptor specificities. In general, CIKs are cytotoxic T lymphocytes (CTL) with the characteristic CD3+CD56+ phenotype.

CIKs can be generated in standard culture conditions in the presence of soluble factors, such as anti-CD3 antibodies, IFN-γ and IL-2. CIK cells can express both T-cell marker CD3 and natural killer cell (i.e., NK) marker CD56, and possess T and/or NK cell phenotypes. CIKs-based therapy became a promising cancer treatment mostly because CIK expansion is relatively easy, and CIKs have anti-tumor activity of T and NK cells without being restricted by the Major Histocompatibility Complex (i.e., MHC).

Generation and expansion of CIK cells are well known to a person of ordinary skill in the art. For instance, isolated T-cells are activated by stimulation with a soluble or immobilized anti-CD3 antibody ex vivo. The isolated cells are then expanded ex vivo by culture with low doses of IL-2 or IL-7 and IL-15, in the absence of exogenous growth factors or accessory cells.

T-cells can be activated by contacting ex vivo with soluble anti-CD3 antibodies or anti-CD3 antibodies immobilized on a solid/insoluble support. In some embodiments, the anti-CD3 antibody is OKT3 (muromonab-CD3) available from Ortho-Biotech (Raritan, N.J.), or monoclonal antibody G19-4 available from Bristol-Meyers Squibb.

The NK-92 cell line is a unique cell line that was discovered to proliferate in the presence of interleukin 2 (IL-2). Gong et al., Leukemia 8:652-658 (1994). These cells have high cytolytic activity against a variety of cancers. The NK-92 cell line is a homogeneous cancerous NK cell population having broad anti-tumor cytotoxicity with predictable yield after expansion. Phase I clinical trials have confirmed its safety profile.

The NK-92 cell line is found to exhibit the CD56^{bright}, CD2, CD7, CD11a, CD28, CD45, and CD54 surface markers. It furthermore does not display the CD1, CD3, CD4, CD5, CD8, CD10, CD14, CD16, CD19, CD20, CD23, and CD34 markers. Growth of NK-92 cells in culture is dependent upon the presence of recombinant interleukin 2 (rIL-2), with a dose as low as 1 IU/mL being sufficient to maintain proliferation. IL-7 and IL-12 do not support long-term growth, nor do other cytokines tested, including IL-1α, IL-6, tumor necrosis factor α, interferon α, and interferon γ. NK-92 has high cytotoxicity even at a low effector:target (E:T) ratio of 1:1. Gong, et al., supra. NK-92 cells are deposited with the American Type Culture Collection (ATCC), designation CRL-2407.

Heretofore, studies on endogenous NK cells have indicated that IL-2 (1000 IU/mL) is critical for NK cell activation during shipment, but that the cells need not be maintained at 37° C. and 5% carbon dioxide. Koepsell, et al., Transfusion 53:398-403 (2013). However, endogenous NK cells are significantly different from NK-92 cells, in large part because of their distinct origins: NK-92 is a cancer-derived cell line, whereas endogenous NK cells are harvested from a donor (or the patient) and processed for infusion into a patient. Endogenous NK cell preparations are heterogeneous cell populations, whereas NK-92 cells are a homogeneous, clonal cell line. NK-92 cells readily proliferate in culture while maintaining cytotoxicity, whereas endogenous NK cells do not. In addition, an endogenous heterogeneous population of NK cells does not aggregate at high density.

In some embodiments, NK-92 cells is administered in a composition comprising NK-92 cells and a medium, such as human serum or an equivalent thereof. In some embodiments, the medium comprises human serum albumin. In some embodiments, the medium comprises human plasma. In some embodiments, the medium comprises about 1% to about 15% human serum or human serum equivalent. In some embodiments, the medium comprises about 1% to about 10% human serum or human serum equivalent. In some embodiments, the medium comprises about 1% to about 5% human serum or human serum equivalent. In a preferred embodiment, the medium comprises about 2.5% human serum or human serum equivalent. In some embodiments, the serum is human AB serum. In some embodiments, a serum substitute that is acceptable for use in human therapeutics is used instead of human serum. Such serum substitutes may be known in the art, or developed in the future. Although concentrations of human serum over 15% can be used, it is contemplated that concentrations greater than about 5% will be cost-prohibitive.

Modified NK-92 cells include but are not limited to those described in, e.g., U.S. Pat. Nos. 7,618,817; 8,034,332; and 8,313,943; and US Patent Application Publication No. 2013/0040386, all of which are incorporated herein by reference in their entireties, such as wild type NK-92, NK-92-CD16, NK-92-CD16-γ, NK-92-CD16-ζ, NK-92-CD16(F157V), NK-92mi and NK-92ci. The NK-92, NK-92mi and NK-92ci cell lines are deposited with the American Type Culture Collection under Deposit Numbers CRL-2407, CRL-2408 and CRL-2409, respectively.

NK-92 cells can be administered to such an individual by absolute numbers of cells, e.g., said individual can be administered from about 1000 cells/injection to up to about 10 billion cells/injection, such as at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) NK-92 cells per injection, or any ranges between any two of the numbers, end points inclusive. In other embodiments, NK-92 cells can be administered to such an individual by relative numbers of cells, e.g., said individual can be administered about 1000 cells to up to about 10 billion cells per kilogram of the individual, such as at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) NK-92 cells per kilogram of the individual, or any ranges between any two of the numbers, end points inclusive. NK-92 cells can also be administered to such a patient according to an approximate ratio between a number of NK-92 cells and the size of the tumor in said patient. The size of the tumor can be determined or estimated by conventional imaging methods, such X-ray, ultrasound imaging, or the like. In other embodiments, the total dose may calculated by m² of body surface area, including 1×10¹¹, 1×10¹⁰, 1×10⁹, 1×10⁸, 1×10⁷, per m². The average person is 1.6-1.8 m².

The NK-92 cells, and optionally other anti-tumor agents, can be administered once to a patient having a solid tumor or can be administered multiple times, e.g., once every 1, 2, 3, 4, 5, 6, 7, 8, 9, 10, 11, 12, 13, 14, 15, 16, 17, 18, 19, 20, 21, 22 or 23 hours, or once every 1, 2, 3, 4, 5, 6 or 7 days, or once every 1, 2, 3, 4, 5, 6, 7, 8, 9, 10 or more weeks during therapy, or any ranges between any two of the numbers, end points inclusive.

In one aspect, a method is provided for killing residual, or remnant, cancer cells in a patient, wherein the patient is recovering from a treatment for a solid tumor, in which the method comprises administering to the patient one or more doses of NK-92 cells sufficient to kill all or substantially all of the remnant cancer cells remaining in the patient. In various embodiments, secondary therapies involve the administration of NK-92 cells to a patient after the patient has undergone treatment under conventional therapy, wherein the administration of NK-92 cells can prevent the maintenance and/or development of remnant cancer cells, including aberrant and recalcitrant cancer stem cells.

For example, prior to administering the NK-92 cells to the patient, said remnant cancer cells may be present in the patient at a level that is less than 20%, 10%, 5% or 1% of the level of cancer cells in a tumor that was detected in the patient prior to the treatment for the tumor.

In some embodiments of the method, said remnant cancer cells comprise cancer stem cells.

### Combinations

In some embodiments of the method, said treatment for tumor includes a conventional therapy, such as chemotherapy, radiotherapy, or hormone treatment, and said remnant cancer cells were and remain substantially resistant to the conventional therapy.

In a further embodiment, the cell compositions of the present invention are administered to a patient in conjunction with (e.g., before, simultaneously or following) conventional therapies, such as chemotherapy agents. For example, in one embodiment, subjects may undergo standard treatment with high dose chemotherapy followed by an infusion of NK-92 cells according to the invention.

Examples of conventional chemotherapeutic agents include, but are not limited to, doxorubicin, cis-platin, fluorouracil, actinomycin, all-trans retinoic acid, azacitidine, azathioprine, bleomycin, bortezomib, carboplatin, capecitabine, chlorambucil, cyclophosphamide, cytarabine, daunorubicin, docetaxel, doxifluridine, epirubicin, epothilone, etoposide, gemcitabine, hydroxyurea, idarubicin, imatinib, irinotecan, mechlorethamine, mercaptopurine, methotrexate, mitxantrone, oxaliplatin, paclitaxel, pemetrexed, teniposide, tioguanine, topotecan, valrubicin, vinbalstine, vincristine, vindesine, and vinorelbine.

In one embodiment, the method is for treating a brain tumor comprising delivering an anti-tumor immunotherapeutic agent directly into the microvasculature of the brain tumor in combination with administration of a conventional antitumor therapy, such as one or more anti-tumor chemoagents, radiation therapy, and/or hormonal therapy. Table 1 lists a number of conventional anti-tumor agents suitable for treating brain tumors as published by the University of California at Los Angeles.

TABLE 1
Anti-neoplastic agents suitable for treating brain tumors

| | | |
|---|---|---|
| 5FC | Accutane Hoffmann-La Roche | AEE788 Novartis |
| AMG-102 | Anti Neoplaston | AQ4N (Banoxantrone) |
| AVANDIA (Rosiglitazone Maleate) | Avastin (Bevacizumab) | BCNU |
| | Genetech | |
| BiCNU Carmustine | Carboplatin | CCI-779 |
| CCNU | CCNU Lomustine | Celecoxib (Systemic) |
| Chloroquine | Cilengitide (EMD 121974) | Cisplatin |
| CPT-11 (CAMPTOSAR, Irinotecan) | Cytoxan | Dasatinib (BMS-354825, Sprycel) |
| Dendritic Cell Therapy | Etoposide (Eposin, Etopophos, | GDC-0449 |
| | Vepesid) | |
| Gleevec (imatinib mesylate) | GLIADEL Wafer | Hydroxychloroquine |
| Hydroxyurea | IL-13 | IMC-3G3 |
| Immune Therapy | Iressa (ZD-1839) | Lapatinib (GW572016) |
| Methotrexate for Cancer (Systemic) | Novocure | OSI-774 |
| PCV | Procarbazine | RAD001 Novartis (mTOR inhibitor) |
| Rapamycin (Rapamune, Sirolimus) | RMP-7 | RTA 744 |
| Simvastatin | Sirolimus | Sorafenib |
| SU-101 | SU5416 Sugen | Sulfasalazine (Azulfidine) |
| Sutent (Pfizer) | Tamoxifen | TARCEVA (erlotinib HCl) |
| Taxol | TEMODAR Schering-Plough | TGF-B Anti-Sense |
| Thalomid (thalidomide) | Topotecan (Systemic) | VEGF Trap |
| VEGF-Trap | Vincristine | Vorinostat (SAHA) |
| XL 765 | XL184 | XL765 |
| Zarnestra (tipifarnib) | ZOCOR (simvastatin) | |

In embodiments in which the immunotherapeutic agent, such as NK-92 cells and a conventional anti-tumor agent such as an immunomodulatory compound or thalidomide are used together, the conventional anti-tumor agent, and immunotherapeutic agent, can be administered to the individual together, e.g., in the same formulation and delivered directly to the solid mass tumor by the method of this invention; separately, e.g., in separate formulations, at approximately the same time; or can be administered separately, e.g., on different dosing schedules or at different times of the day. When administered separately, the conventional anti-tumor agent can be administered in any suitable route, such as intravenous or oral administration. The immunotherapeutic agent, such as natural killer cells, e.g., PINK cells, pools and/or combinations of the same can be administered without regard to whether the immunotherapeutic agent and/or conventional anti-tumor therapies have been administered to the individual in the past.

All publications or references cited in the present specification are hereby incorporated by reference.

### Example

The following example illustrates how microinjection can be achieved by the methods described herein.

NK-92 cells (wild type-ATCC® CRL2704™) are isolated from their growth medium and subjected to sufficient irradiation to render the cells incapable of proliferation and to have a finite life span. These cells are loaded into a syringe connected to a Rebar® catheter available from ev3, Irvine, Calif. The distal end of the catheter is introduced into the femoral artery by conventional means and directed into the microvasculature of a prostate tumor in a male patient by conventional fluoroscopy. Once the distal end of the catheter is so positioned, the clinician administers the NK-92 cells into the microvasculature leading into the tumor to effect microinjection of these cells so as to directly contact the tumor. The cells are preferably maintained in a suitable medium such as an aqueous medium containing IL-2. Sufficient number of cells are delivered to provide a therapeutic amount. Afterwards, the catheter is removed by conventional means and the femoral artery wound is closed again by conventional means.

What is claimed is:

1. A method to treat a solid tumor by the direct infusion of an anti-tumor immunotherapeutic agent into said tumor which method comprises:
a) placing a microcatheter into or proximate one or more of the microvasculature arteries feeding said tumor;
b) infusing through said microcatheter said anti-tumor immunotherapeutic agent so that said agent is directed by the microvasculature into said tumor; and
c) maintaining said infusion until a sufficient amount of said anti-tumor agent has been infused so as to treat said tumor.

2. The method of claim 1 wherein said anti-tumor agent is selected from anti-tumor antibodies and anti-tumor cells.

3. The method of claim 2 wherein said anti-tumor immunotherapeutic agent comprises cytolytic NK-92 cells.

4. The method of claim 2 wherein said immunotherapeutic agent comprises T-cells.

5. The method of claim 2 wherein said immunotherapeutic agent comprises modified cytolytic NK-92 cells.

6. The method of claim 2 wherein said immunotherapeutic agent comprises NK-92 cells selected from the group consisting of NK-92, NK-92-CD16, NK-92-CD16-γ, NK-92-CD16-ζ, NK-92-CD16(F157V), NK-92mi and NK-92ci.

7. The method of claim 4 wherein said immunotherapeutic agent comprises cytolytic T-cells.

8. The method of claim 2 wherein said immunotherapeutic agent comprises cytolytic NK-92 cells and/or T-cells complexed to an antibody wherein said NK-92 cells and T-cells express a surface epitope recognized by the antibody.

9. The method of claim 1, further comprising placing a balloon into or proximate one or more of the microvasculature arteries feeding said tumor and inflating the balloon, such that said agent is retained in the tumor for a set period of time.

10. A method to treat a solid brain tumor by the direct infusion of an anti-tumor immunotherapeutic agent into said tumor which method comprises:
a) placing a microcatheter into or proximate one or more of the microvasculature arteries feeding said tumor;
b) infusing through said microcatheter said anti-tumor immunotherapeutic agent so that said agent is directed by the microvasculature into said tumor; and
c) maintaining said infusion until a sufficient amount of said anti-tumor immunotherapeutic agent has been infused so as to treat said tumor.

11. The method of claim 10 wherein said anti-tumor immunotherapeutic agent comprises or expresses chimeric antigen receptors specific for an antigen expressed by the tumor.

12. The method of claim 10 wherein said anti-tumor immunotherapeutic agent comprises NK-92 cells.

13. The method of claim 10, further comprising placing a balloon into or proximate one or more of the microvasculature arteries feeding said tumor and inflating the balloon, such that said agent is retained in the tumor for a set period of time.

14. Use of a an anti-tumor therapeutic agent comprising NK-92 cells in a direct infusion system to treat a solid tumor by the direct infusion of NK-92 cells into said tumor where the direct infusion comprises:
a) placing a microcatheter into or proximate one or more of the microvasculature arteries feeding said tumor;
b) infusing through said microcatheter said NK-92 cells so that said NK-92 cells are directed by the microvasculature into said tumor; and
c) maintaining said infusion until a sufficient amount of said NK-92 cells have been infused so as to treat said tumor.

15. The use of claim 14 wherein said anti-tumor agent further comprises the use of antibodies and anti-tumor cells.

16. The use of claim 14 wherein said anti-tumor immunotherapeutic agent comprises cytolytic NK-92 cells.

17. The use of claim 15 wherein said immunotherapeutic agent comprises T-cells.

18. The use of claim 14 wherein said immunotherapeutic agent comprises modified cytolytic NK-92 cells.

19. The use of claim 14 wherein said immunotherapeutic agent comprises NK-92 cells selected from the group consisting of NK-92, NK-92-CD16, NK-92-CD16-γ, NK-92-CD16-ζ, NK-92-CD16(F157V), NK-92mi and NK-92ci.

20. The use of claim 17 wherein said immunotherapeutic agent comprises cytolytic T-cells.

21. The use of claim 15 wherein said immunotherapeutic agent comprises cytolytic NK-92 cells and/or T-cells complexed to an antibody wherein said NK-92 cells and T-cells express a surface epitope recognized by the antibody.

22. The use of claim 14, further comprising the use of a balloon that can be placed into or proximate one or more of the microvasculature arteries feeding said tumor, which balloon can be inflated, such that said agent is retained in the tumor for a set period of time.

23. Use of an anti-tumor immunotherapeutic agent with a direct infusion system to treat a solid brain tumor by the direct infusion of an anti-tumor immunotherapeutic agent into said tumor which use comprises:
a) placing a microcatheter into or proximate one or more of the microvasculature arteries feeding said tumor;
b) infusing through said microcatheter said anti-tumor immunotherapeutic agent so that said agent is directed by the microvasculature into said tumor; and
c) maintaining said infusion until a sufficient amount of said anti-tumor immunotherapeutic agent has been infused so as to treat said tumor.

24. The use of claim 23 wherein said anti-tumor immunotherapeutic agent comprises or expresses chimeric antigen receptors specific for an antigen expressed by the tumor.

25. The use of claim 23 wherein said anti-tumor immunotherapeutic agent comprises NK-92 cells.

26. The use of claim 23, further comprising the use of a balloon that is or can be placed into or proximate one or more of the microvasculature arteries feeding said tumor and inflated, such that said agent is retained in the tumor for a set period of time.
